(19) United States
(12) Patent Application Publication
(10) Pub. No.: US 2014/0187888 A1
(43) Pub. Date: Jul. 3, 2014

(54) MONITORING AND COMMUNICATION SYSTEM FOR PATIENTS WITH DIABETES

(71) Applicant: TYPE2COMPANION.COM, LLC, Seattle, WA (US)

(72) Inventor: George A. Hatziantoniou, Seattle, WA (US)

(73) Assignee: TYPE2COMPANION.COM, LLC, Seattle, WA (US)

(21) Appl. No.: 14/141,754

(22) Filed: Dec. 27, 2013

Related U.S. Application Data
(60) Provisional application No. 61/746,489, filed on Dec. 27, 2012.

(51) Int. Cl. A61B 5/00; A61B 5/021; A61B 5/145

(52) U.S. Cl. CPC A61B 5/0022; A61B 5/14532; A61B 5/021
USPC 600/365; 600/300; 600/485

(57) ABSTRACT

A method of facilitating communications between a patient and a health care provider is provided. The method includes: receiving a patient test result at a mobile computing device; transmitting the patient test result from a wireless interface of the mobile computing device; receiving the patient test result at a host computing system; comparing the received patient test result with a predetermined threshold; and if the patient test result exceeds the predetermined threshold, transmitting an alert to a health care provider.

### CROSS-REFERENCE TO RELATED APPLICATIONS

This application claims the benefit of U.S. Provisional Application No. 61/746,489, filed Dec. 27, 2012, the disclosure of which is incorporated herein by reference in its entirety.

### BACKGROUND

Diabetes mellitus, commonly referred to generally as diabetes, is a group of diseases characterized by high blood glucose levels that result from defects in the body's ability to produce and/or use insulin. Type 2 diabetes is the most common form of diabetes. In type 2 diabetes, either the body does not produce enough insulin or the cells ignore the insulin.

Monitoring and managing blood glucose is an important tool for diabetes control. Patients may use blood glucose monitors to measure their glucose levels and are typically advised by their health care provider to maintain a record of their glucose measurements over a period of time. This is typically performed by manually writing each measurement into a logbook.

### SUMMARY

In accordance with aspects of the present invention, a communication system is provided for recording a patient's glucose levels and communicating with the patient's health care provider.

Other features and aspects of the invention will become apparent from the following detailed description, taken in conjunction with the accompanying drawings which illustrate, by way of example, the features in accordance with embodiments of the invention.

### DESCRIPTION OF THE DRAWINGS

FIG. 1 illustrates an exemplary communications system in which embodiments of the present invention may be implemented.

FIGS. 2-9 illustrate various web page interfaces in accordance with embodiments of the present invention.

FIGS. 10A-10I show screen shots of the patient or physician interface in accordance with embodiments of the present invention.

FIG. 11 is a flow chart illustrating communications between the patient and health care providers, in accordance with embodiments of the present invention.

FIG. 12 is a flow chart illustrating the communication between the medical staff and the patient, in accordance with embodiments of the present invention.

FIG. 13 shows an exemplary process flow to be used for a mobile interface application, in accordance with embodiments of the present invention.

### DETAILED DESCRIPTION

In accordance with embodiments of the present inventions, a communications system is provided to facilitate and expedite physician and patient communications and interactions. In a preferred embodiment, the communications system includes one or more patient interfaces configured to run on a general purpose computer or a mobile computing device, such as a smartphone, tablet computer, or laptop. The mobile computing device may be, for example, a smartphone or tablet running the iOS operating system by Apple Inc. of Cupertino, Calif., or the Android operating system by Google Inc. of Mountain View, Calif. The patient interface may be implemented as a dedicated patient interface application running on the mobile computing device operating system. Alternatively, the patient interface may be provided via a web browser application running on any computing device.

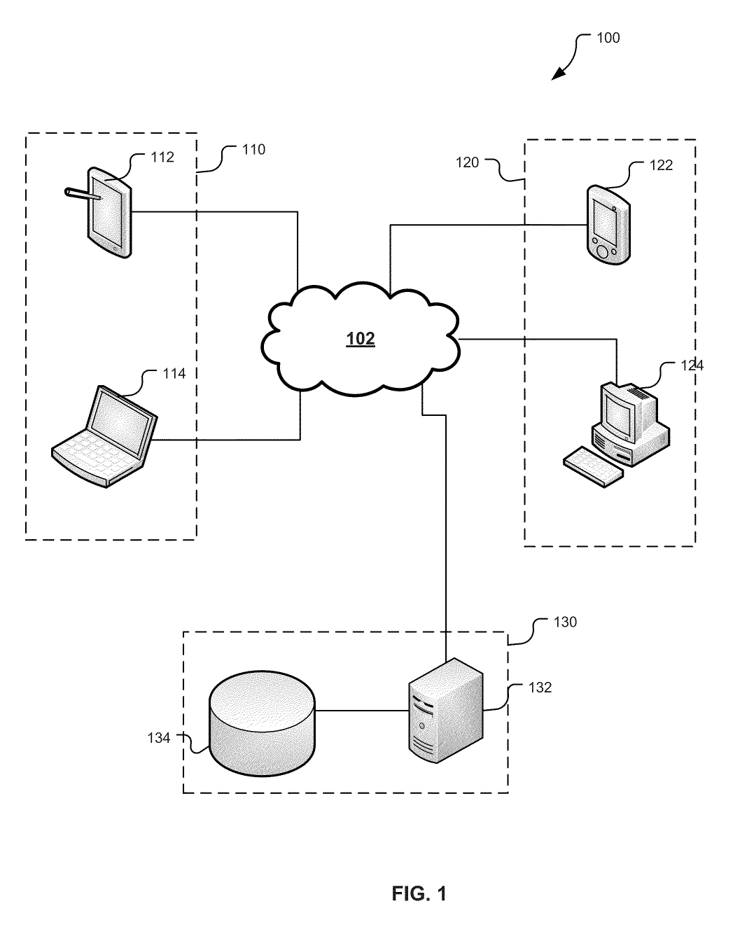

FIG. 1 illustrates an exemplary communications system 100 in which embodiments of the present invention may be implemented. Patient devices 110 may be used by the patient to access the system for inputting the patient's health data and other patient information. The patient devices 110 may comprise, e.g., a smartphone 112 or a laptop computer 114, running dedicated patient interface applications or a web browser accessing a web page providing a patient interface. Physician devices 120 may be used by the health care provider (e.g., physician, nurse, counselor, or other health care practitioner) to access the system for reviewing the patient's health data and other patient information. The physician devices 120 may comprise, e.g., a smartphone 122 or a laptop computer 124, running dedicated physician interface applications or a web browser accessing a web page providing a physician interface. A host system 130 comprising one or more servers 132 and data storage devices 134 may be used to manage communications between the patient devices 110 and the physician devices 120, and to store data, such as patient health data. All of the various components of the communications system 100 may communicate over the Internet via a variety of known protocols, communication technologies, and network security measures.

The system 100 includes a mobile application and interface to provide enhanced and more efficient exchange of patient test results safely and securely. By improving communication between the patient and health care provider, better and improved patient care with fewer co morbidities in patients with diabetes may be achieved.

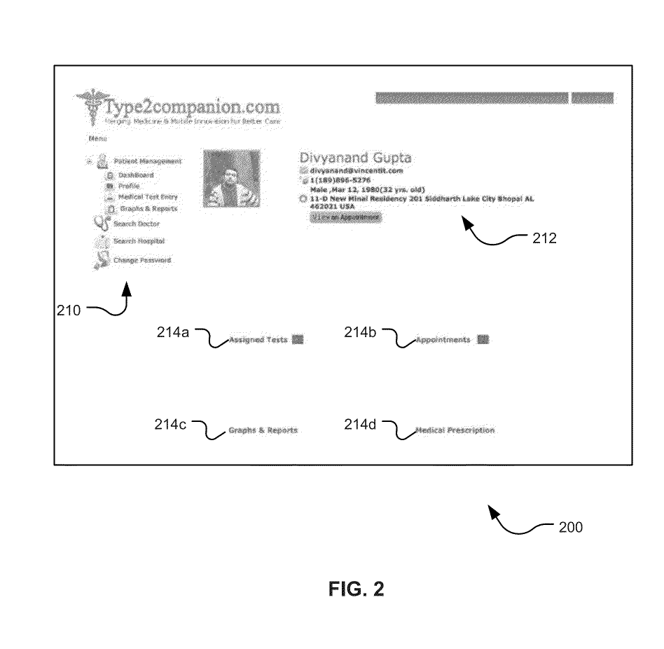

In preferred embodiments, the patients using this system 100 are people with type 2 diabetes with or without hypertension. The patient will program the application per the physician's requests as to when the patient is to monitor blood glucose and/or blood pressure readings, such as readings before meals, after meals, bedtime, fasting etc. FIG. 2 illustrates a web page patient interface Introductory Page 200 for managing the patient account. Section 210 provides links to various features on the patient web site. Section 212 displays patient information, such as name, address, and other contact information.

“Assigned Tests” link 214a retrieves a list of the tests that patient has been instructed to take. This list may be provided by the health care provider and communicated from a physician device 120 to the patient device 110, or may be entered manually.

“Appointments” link 214b retrieves a list of upcoming and/or past appointments with the patient's health care provider. This list may be manually entered or dynamically updated with information from the health care provider.

“Graphs & Reports” link 214c provides a link to various graphs and reports for the patient. This may include, for example: (1) Blood Glucose, (a) fasting, (b) before meals, (c) after meals, and (d) at bedtime; (2) Systolic Blood Pressure; (3) Diastolic Blood Pressure; (4) Calories Utilized During Physical Exercise; and (5) Heart Rate. Other graphs and reports containing information useful for treating or managing diabetes may also be used.

“Medical Prescriptions” link 214d provides a link to a list of the patient's prescriptions. This list may be manually entered or dynamically updated with information from the health care provider.

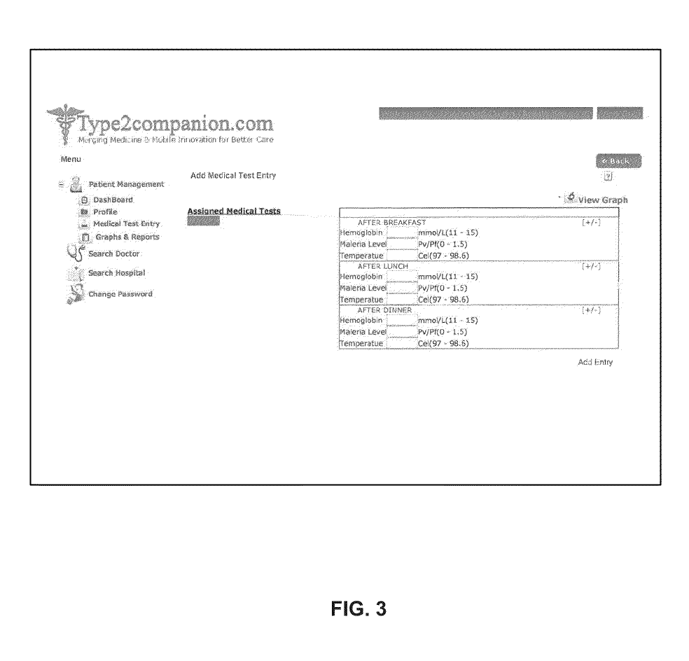

FIG. 3 shows a table of test results entered by the medical staff for patients who are seen in the physician's office.

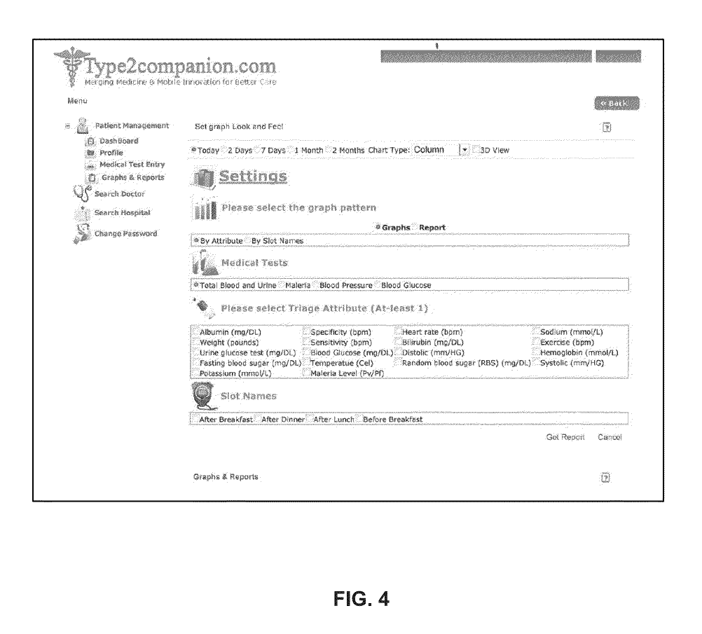

FIG. 4 shows a patient/physician discussion and decision regarding which daily test is to be performed by the patient and entered into the mobile device for storage and later retrieval by the health care provider.

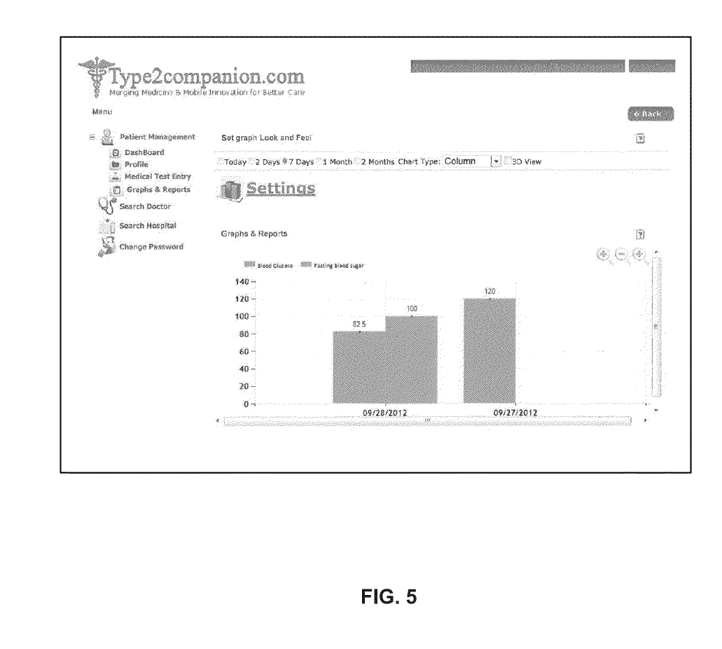

FIG. 5 shows a graphical analysis upon mobile request to retrieve a patient's test results based on the decision made in FIG. 4.

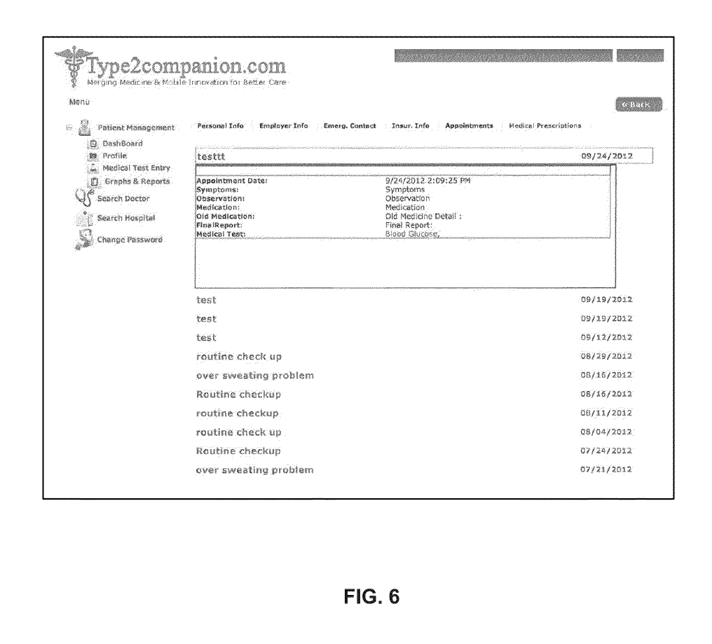

FIG. 6 shows an exemplary patient mobile application Dashboard. This Dashboard interface can be used by the patient to enter all medications being taken by the patient, side effects, insurance information, and/or other information related to the patient's health, treatment, and/or management.

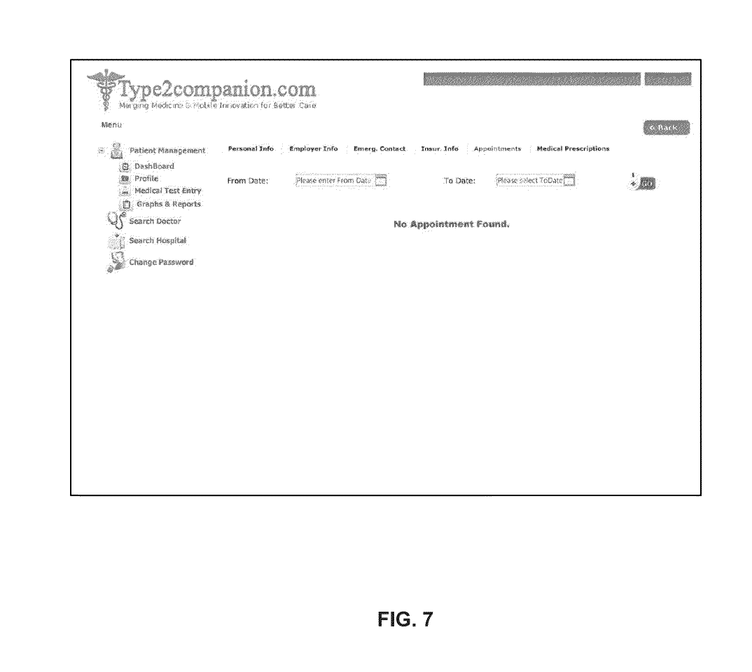

FIG. 7 shows an appointment screen where a patient can search for upcoming and/or past appointments with health care providers.

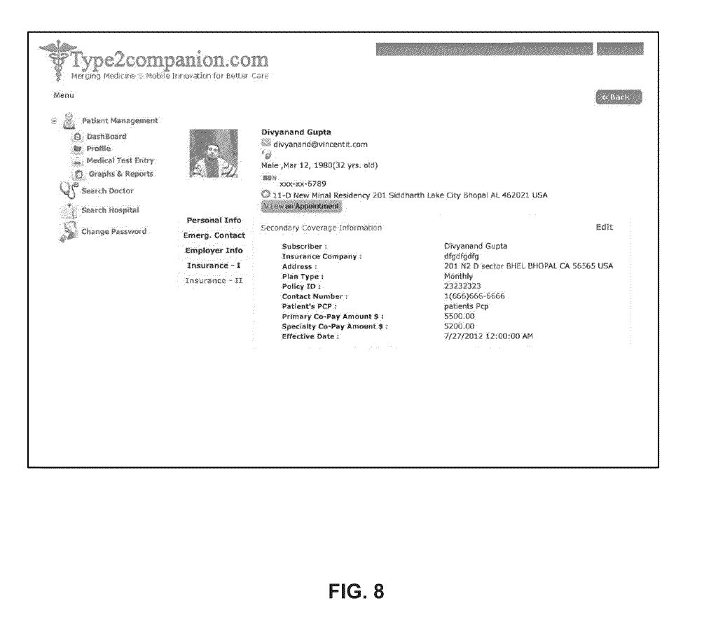

FIG. 8 shows a patient information screen containing patient identification information, insurance information, and/or other information that may be used by the health care provider.

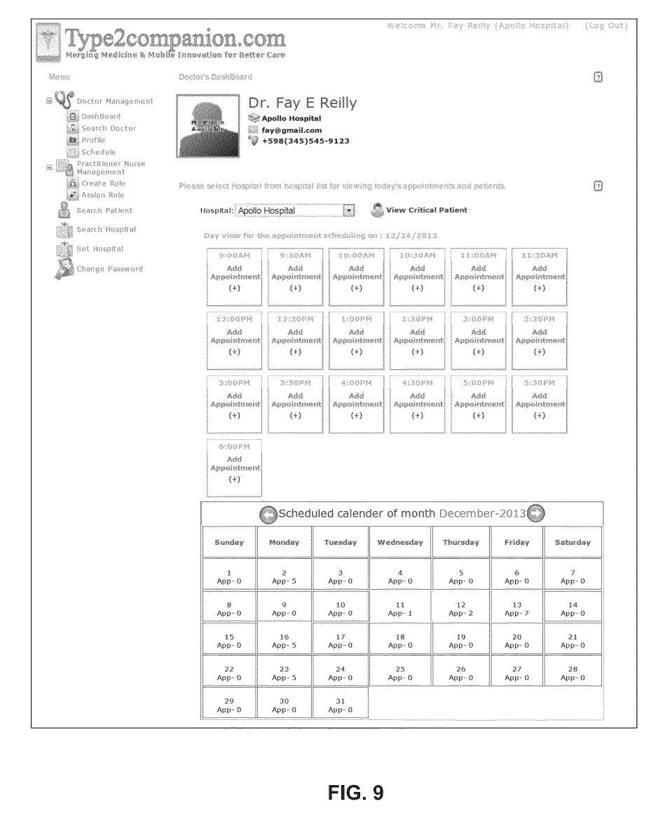

FIG. 9 shows an exemplary mobile application physician or other health care provider Mobile Dashboard. The Mobile Dashboard can provide an interface for the health care provider to retrieve information useful for patient management, such as a mobile calendar displaying appointments, mobile notifications indicating abnormal or dangerous test results for patients that warrant immediate attention, a mobile directory providing access to contact information for patients, and a mobile chart login for reviewing a patient's health chart.

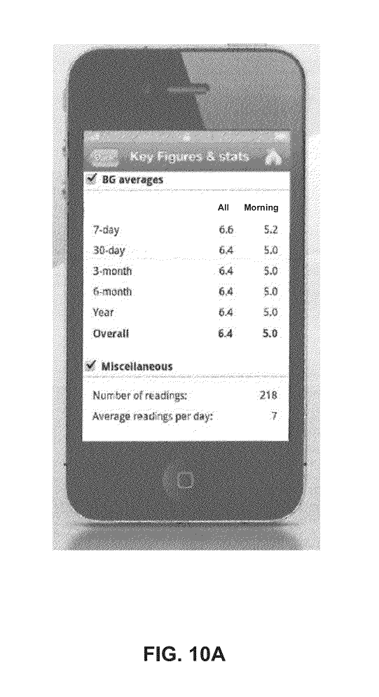

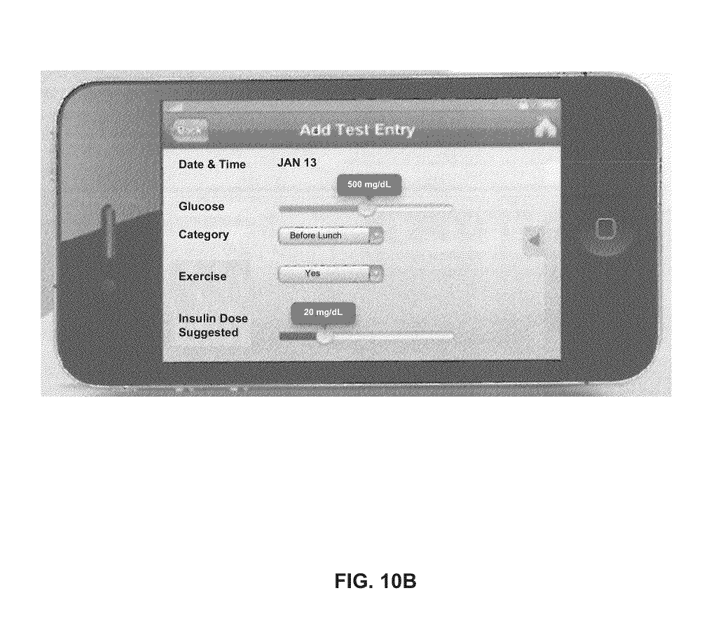

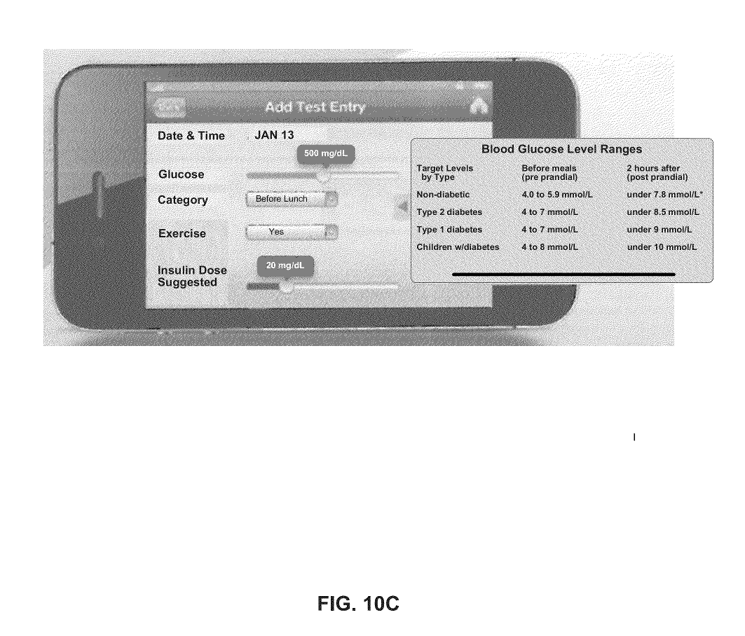

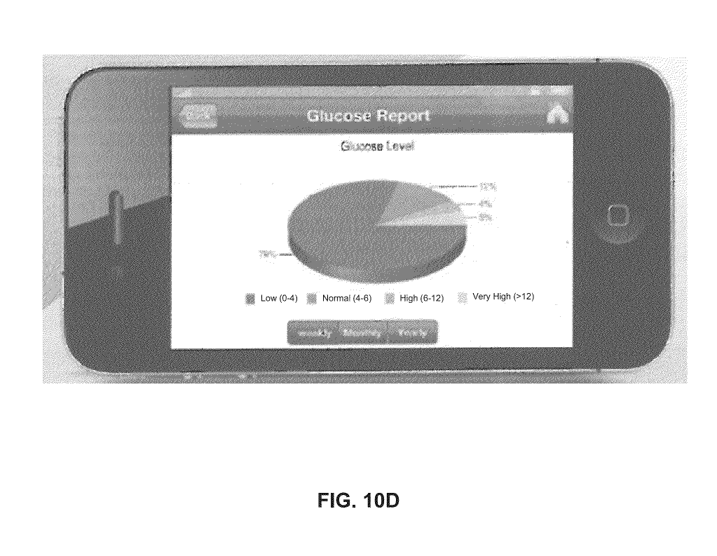

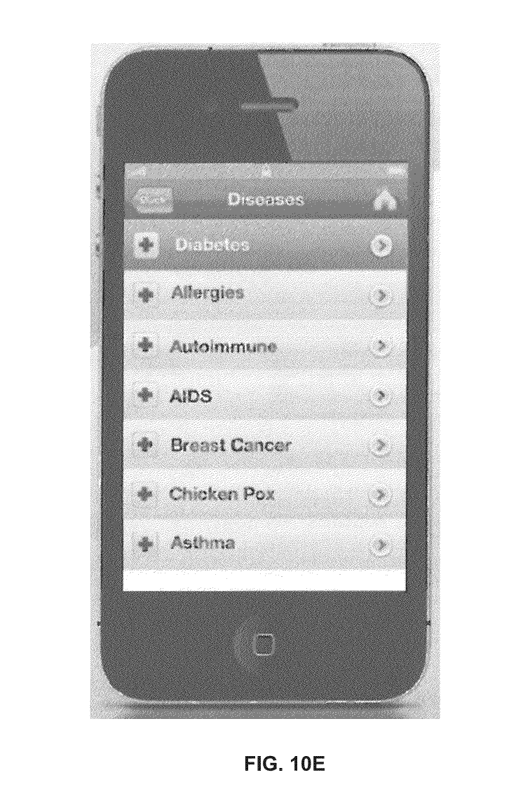

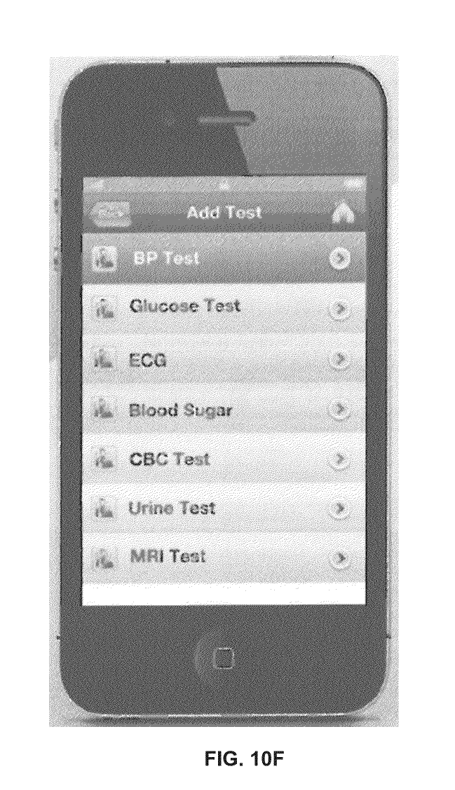

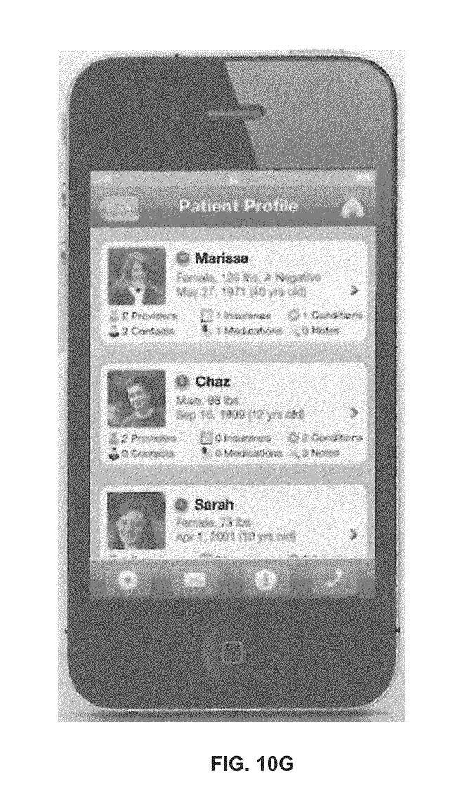

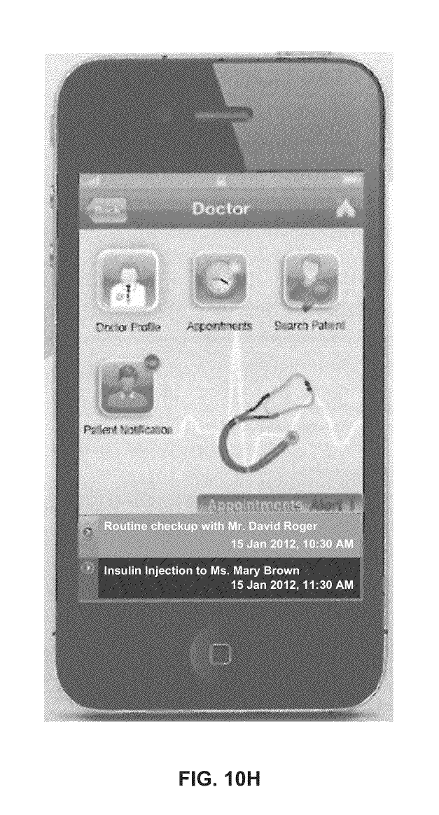

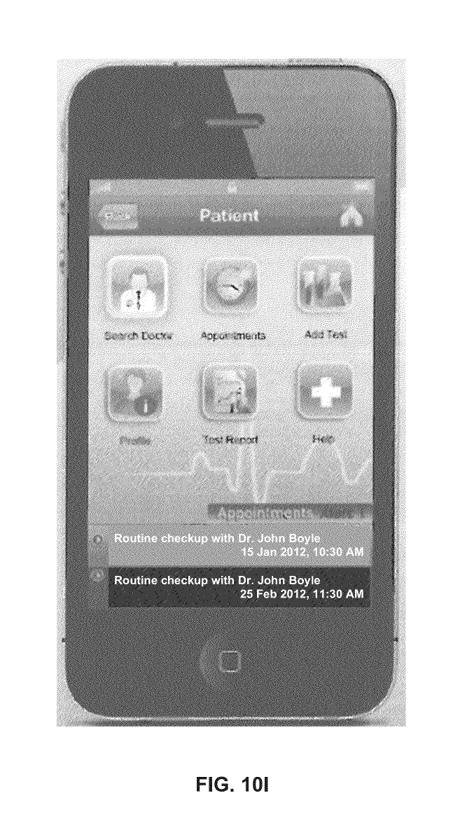

FIGS. 10A-10I show screen shots of various screens available to a patient and/or physician for accessing the patient or physician interface via an application on a smartphone.

FIG. 10A shows a patient or physician interface application displaying blood glucose test result averages and individual daily test results. The averages may be computed based on different intervals, such as a 7 day average, 30 day average, 60 day average, 90 day average, etc.

FIGS. 10B-10C show a patient data entry screen for entering patient data for storage and future review by the patient or health care provider. After the data is entered into the mobile computing device, it is transmitted to the host system 130 for storage in storage device 134. The host system 130 may also transmit this data to a mobile patient chart for review by health care providers.

FIG. 10D shows a mobile report based on inputted patient test results and other entries into the mobile patient interface. The data may be displayed in graphical form for easier review. As shown in FIG. 10C, the data is displayed as a pie chart indicating percentages of test results that were abnormal or normal, or various percentages of times of varying degrees of abnormal test results.

FIG. 10E shows an exemplary “Mobile Hospital” function that may be used to monitor other disease states, such as asthma, AIDS, etc.

FIG. 10F shows an exemplary interface for selecting test results and reports for the patient. This interface may be used by the patient and/or health care provider to retrieve, for example, reports of blood and urine samples taken in the physician's office.

FIG. 10G shows a Contacts screen for the physician interface. This Contacts screen may be used by the health care provider to retrieve patient contact information, patient photos, or other patient information remotely on the health care provider's mobile device. This interface may also provide a link to retrieve patient mobile charts, thereby providing the health care provider the ability to access patient records remotely at any time.

FIG. 10H shows a physician's mobile user login screen.

FIG. 10I shows a patient's mobile user login screen.

In accordance with embodiments of the present invention, the patient may access the patient interface via a mobile device to manually enter blood glucose and/or blood pressure measurements. These measurements may have been made by the patient using at-home glucose monitors and blood pressure monitors, or may be made by a health care provider in the clinic. Whether the patient data is entered on a laptop, desktop, notebook computer or smartphone, the readings are then securely stored in the application for future review by the physician in a mobile check-up with the patient.

The application provides a mechanism within the interface itself where the patient and physician have predetermined thresholds that are considered dangerous to the patient's health. When the patient's blood glucose or blood pressure readings exceed the predetermined thresholds, the physician is immediately notified via the mobile interface or other communication method, such as by text message, e-mail, or telephone call. At that point the physician will then instruct the patient accordingly, thus helping avoid any further medical complications. The patient's drug protocol can then be reviewed and changes can be made to stabilize the patient and improve patient care.

A patient can save and store past blood glucose and blood pressure readings. In some embodiments, the system may provide an unlimited storage of historical measurements. Numerical averages can be determined and bar graphs can be shown in three dimensional viewings. The “mobile graphs” can be broken down into daily readings or specific readings such as fasting blood glucose, or bedtime glucose readings which will help the physician upon the patient's “mobile review” if there is a specific problem at a specific time of the day and isolate that problem and treat the problem accordingly. The “mobile graph” can be done for many different time periods whether daily, weekly, monthly, etc.

The patient's health care provider can use the physician interface at any time to safely and securely log into the patient's health recorder to retrieve the patient's health measurements via a “mobile chart” for a “mobile review” of the patient's recent health history. The health care provider may then determine whether the treatment protocol that has been prescribed to this patient is satisfactory or not.

The prompt and swift intervention of the physician with regard to unsatisfactory treatment is very important for the long term health and well being of the patient, thereby decreasing mortality, morbidity, and healthcare costs. Periodic “mobile chart reviews” at the physician's convenience are recommended.

Upon the mobile chart review the interface will allow the physician and patient to interact and verify and explain any changes in protocol. The physician will have access to the patient's “mobile drug profile” where the patient has safely entered, secured, and saved all prescription and non prescription drugs the patient is taking.

The mobile drug profile may also contain a section for adverse reactions that a patient has encountered when using a particular drug. The interface may also contain mobile reminder of health care provider appointments.

In some embodiments, the system may facilitate real-time communication between the patient and the health care provider. For example, the patient and/or physician interfaces may include a “The Doctor Will See you Now” link which will launch a communications application, such as voice-over-Internet-Protocol application (e.g., Skype from Microsoft Corporation of Redmond, Washington). This communication application may permit a physician, after having reviewed the patient's health information, to communicate directly with the patient in real-time, via voice, video, and/or text.

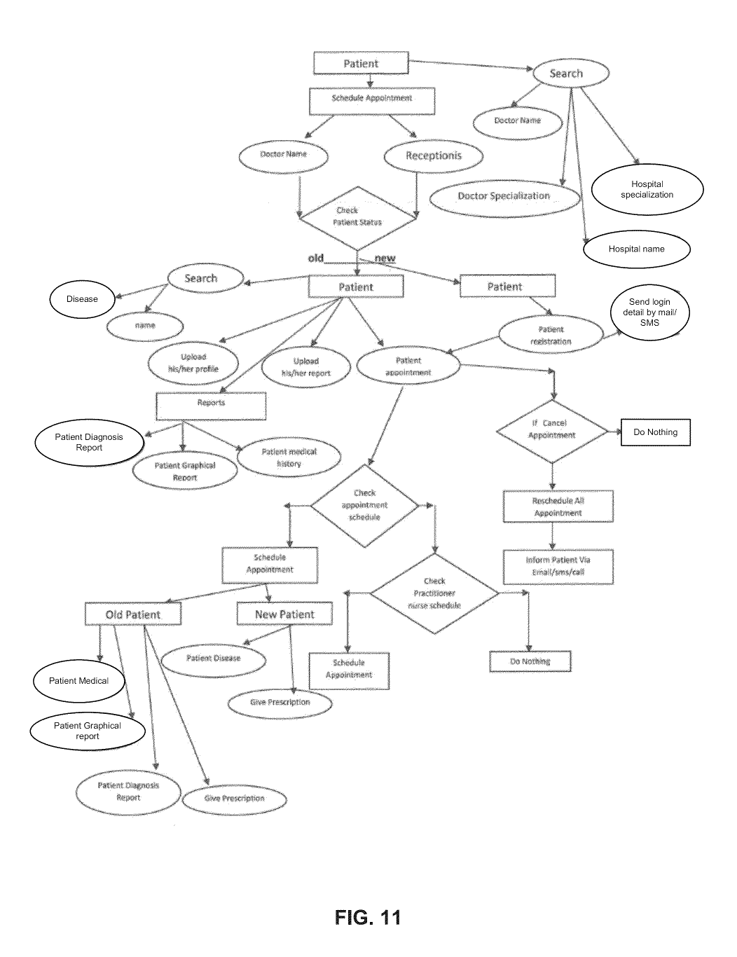

FIG. 11 is a flow chart illustrating communications between the patient and health care providers, in accordance with embodiments of the present invention. The process begins with the patient scheduling on appointment via the mobile/web application to find the physician at the particular facility that physician is affiliated with and his schedule. This interaction can take place via the physician's medical assistant or receptionist. A new patient can be registered via a web interface or mobile application interface. All medical information can be provided securely via these interfaces.

The appointment interface on the mobile application will then register appointment and send a reminder to the patient a predetermined period in advance of the appointment (e.g., two days) to confirm the appointment.

A patient who had previously met with this physician will then have his/her history retrieved by the medical staff for review and then sent to the physician electronically for his/her review. The graphical reports and charts of the patient's history of blood glucose and blood pressure readings can be included for medical staff review before the patient's appointment.

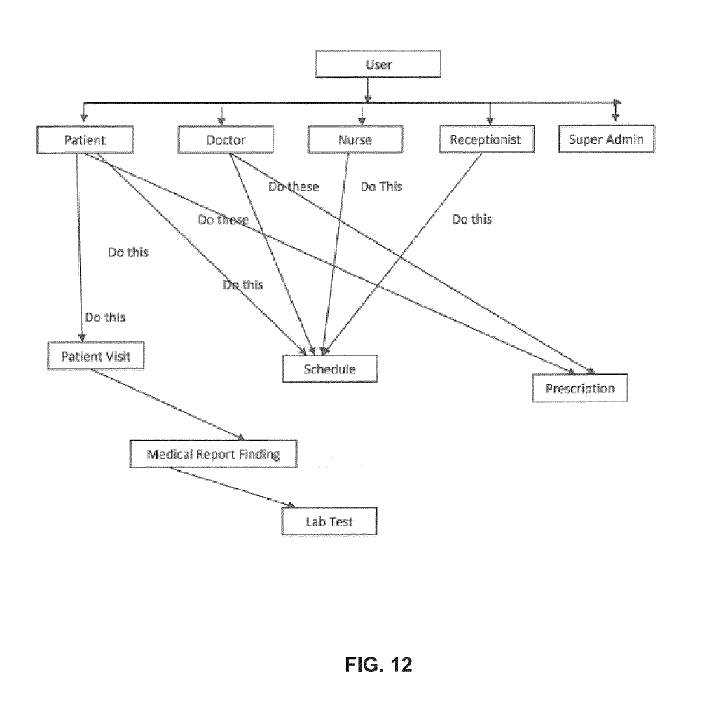

FIG. 12 is a flow chart illustrating the communication between the medical staff and the patient with regard to test results that were obtained after the doctor has seen the patient and all the lab work has been completed. The patient and physician can either schedule on appointment to review results or the medical staff can send the patient his/her results safely and securely via the patient interface. The physician can also use the system to to send the patient any new prescription that might be needed to help the patient with his/her care.

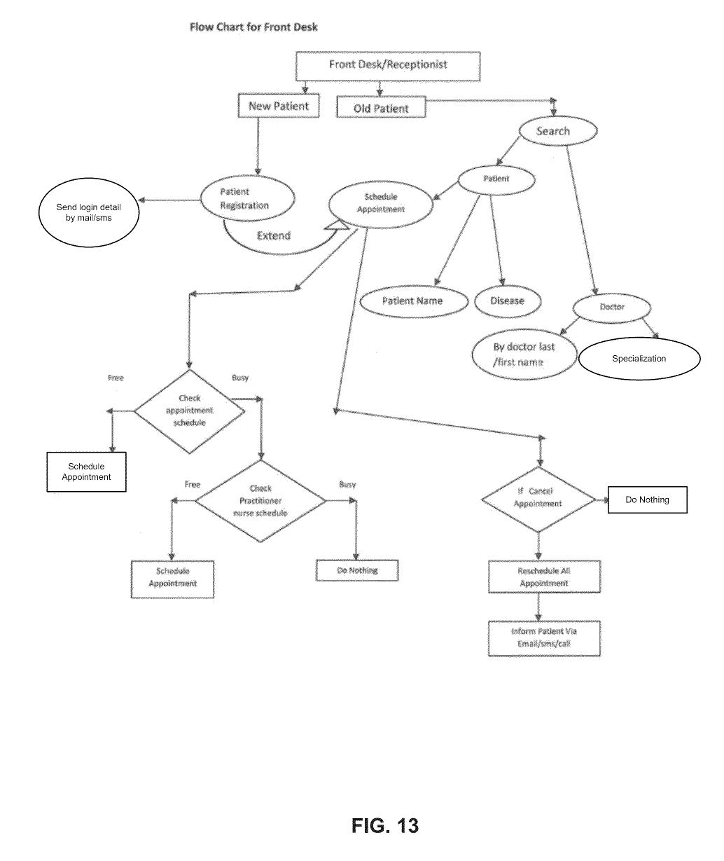

FIG. 13 shows an exemplary process flow to be used for a mobile interface application. According to this process flow, the patient may search for a health care provider, schedule appointments, explain medical conditions, schedule medical tests, etc.

Embodiments of the present invention may provide various advantages not provided by prior art systems. For example, the system stores a history of the patient's health measurements, prescriptions, adverse reactions, and other information pertinent to diabetes treatment. This information may be viewed by the physician or other health care provider at any time. In addition, the patient and/or health care provider may be alerted when the patient's health measurements exceed predetermined levels. Finally, the system may provide improved communication between the patient and health care provider.

In the preceding description, reference is made to the accompanying drawings which illustrate several embodiments of the present invention. It is understood that other embodiments may be utilized and mechanical, compositional, structural, electrical, and operational changes may be made without departing from the spirit and scope of the present disclosure. It should be understood that the invention can be practiced with modification and alteration within the spirit and scope of the claims and the equivalents thereof.

What is claimed is:

1. A method of facilitating communications between a patient and a health care provider, comprising:
receiving a patient test result at a mobile computing device;
transmitting the patient test result from a wireless interface of the mobile computing device;
receiving the patient test result at a computing system;
comparing the received patient test result with a predetermined threshold; and
if the patient test result exceeds the predetermined threshold, transmitting an alert to a health care provider.

2. The method of claim 1, wherein said patient test result comprises a blood glucose level.

3. The method of claim 1, wherein said patient test result comprises a blood pressure level.

4. The method of claim 1, further comprising storing a plurality of patient test results taken at different times.
